(19) United States
(12) Patent Application Publication
(10) Pub. No.: US 2023/0309863 A1
(43) Pub. Date: Oct. 5, 2023

(54) BIOLOGICAL DETECTION DEVICE, BIOLOGICAL DETECTION METHOD, AND PROGRAM

(71) Applicant: Data Solutions, Inc., Tokyo (JP)

(72) Inventors: Tomoaki OTSUKI, Yokohama-shi, Kanagawa (JP); Chen YE, Yokohama-shi, Kanagawa (JP)

(73) Assignee: Data Solutions, Inc., Tokyo (JP)

(21) Appl. No.: 18/043,318

(22) PCT Filed: Aug. 25, 2021
(86) PCT No.: PCT/JP2021/031104

§ 371 Date: Feb. 27, 2023

(30) Foreign Application Priority Data
Aug. 31, 2020 (JP) 2020-145624

(51) Int. Cl. A61B 5/113; A61B 5/08

(52) U.S. Cl. CPC A61B 5/1135; A61B 5/0816

(57) ABSTRACT

A biological detection device includes a signal acquirer that acquires a signal including a respiration component, a filter that attenuates a frequency component other than the respiration component included in the signal and generates a post-processing signal, an analyzer that analyzes the post-processing signal to generate a spectrogram, a variation calculator that calculates variation of energy in a predetermined frequency band in energy of the frequency component indicated by the spectrogram, a first determiner that determines whether the variation is larger than a threshold, a second determiner that, if it is determined by the first determiner that the variation is not larger than the threshold, determines whether a higher harmonic wave with respect to a second peak having energy equal to or more than a predetermined ratio to a first peak where the energy is maximum is present in the predetermined frequency band, and an output unit that outputs a respiration rate based on a frequency of the first peak if it is determined by the second determiner that the higher harmonic wave is absent and outputs a respiration rate based on a fundamental frequency of the higher harmonic wave if it is determined by the second determiner that the higher harmonic wave is present.

### TECHNICAL FIELD

The present invention relates to a biological detection device, a biological detection method, and a program.

### BACKGROUND ART

There has been known a technique for measuring biological information such as a heart rate with wearable equipment and notifying a user if the biological information has an abnormality (for example, Non-Patent Literature 1).

In a watching system, first, observation equipment such as a nurse call button, a human sensor, a Doppler sensor, a heartbeat meter, a respiration measuring instrument, a thermo-camera, a manometer, a clinical thermometer, an illuminance meter, a thermometer, or a hygrometer is connected to an observed person such as an aged person. The watching system acquires observation information of the observed person in this way. The watching system determines, based on the observation information, whether an emergency alarm condition is satisfied and, if an emergency occurs, performs an emergency alarm. There has been known such a watching system that uses a vital sensor (for example, Patent Literature 1).

### CITATION LIST

### Non-Patent Literature

Non-Patent Literature 1: “Heart Rate. Its meaning and a display method in an Apple Watch (registered trademark)”, [online], Jan. 21, 2020, [searched on Mar. 2, 2020], Internet <URL: http://support.apple.com/ja-jp/HT204666>

### Patent Literature

Patent Literature 1: Japanese Patent Application Laid-Open No. 2017-151755

### SUMMARY OF INVENTION

### Technical Problem

In view of the fact that it is difficult to accurately measure respiration by the prior art, an object of the present invention is to accurately measure respiration.

### Solution to Problem

A requirement of a biological detection device is to include:
a signal acquirer that acquires a signal including a respiration component;
a filter that attenuates a frequency component other than the respiration component included in the signal and generates a post-processing signal;
an analyzer that analyzes the post-processing signal to generate a spectrogram;
a variation calculator that calculates variation of energy in a predetermined frequency band in energy of the frequency component indicated by the spectrogram;
a first determiner that determines whether the variation is larger than a threshold;
a second determiner that, if it is determined by the first determiner that the variation is not larger than the threshold, determines whether a higher harmonic wave with respect to a second peak having energy equal to or more than a predetermined ratio to a first peak where the energy is maximum is present in the predetermined frequency band; and
an output unit that outputs a respiration rate based on a frequency of the first peak if it is determined by the second determiner that the higher harmonic wave is absent and outputs a respiration rate based on a fundamental frequency of the higher harmonic wave if it is determined by the second determiner that the higher harmonic wave is present.

### Advantageous Effects of Invention

According to the disclosed technique, it is possible to accurately measure respiration.

### BRIEF DESCRIPTION OF DRAWINGS

FIG. 1 is a diagram showing an overall configuration example in a first embodiment.

FIG. 2 is a diagram showing an example of a Doppler radar.

FIG. 3 is a diagram showing an example of a biological detection device.

FIG. 4 is a diagram showing an overall processing example in the first embodiment.

FIG. 5 is a diagram showing an overall processing example in a second embodiment.

FIG. 6 is a diagram showing a first experiment result.

FIG. 7 is a diagram showing an evaluation result of comparison with a true value in a first experiment.

FIG. 8 is a diagram showing a second experiment example.

FIG. 9 is a diagram showing an evaluation result of comparison with the true value in a second experiment.

FIG. 10 is a diagram showing an occurrence example of apnea.

FIG. 11 is a diagram showing a calculation result of dispersion by a single time window.

FIG. 12 is a diagram showing a calculation result of dispersion by three time windows.

FIG. 13 is a diagram showing an evaluation result of comparison with the true value in an experiment in which the single time window is used.

FIG. 14 is a diagram showing an evaluation result of comparison with the true value in an experiment in which the three time windows are used.

FIG. 15 is a diagram showing a third experiment result.

FIG. 16 is a diagram showing an evaluation result of comparison with the true value in a third experiment.

FIG. 17 is a diagram showing a fourth experiment result.

FIG. 18 is a diagram showing an evaluation result of comparison with the true value in a fourth experiment.

FIG. 19 is a diagram showing a first comparative example.

FIG. 20 is a diagram showing a second comparative example.

FIG. 21 is a diagram showing a functional configuration example.

FIG. 22 is a diagram showing an example of IQ data measured by a Doppler radar.

### DESCRIPTION OF EMBODIMENTS

Optimum and minimum modes for carrying out the invention are explained below with reference to the drawings. Note that, in the drawings, when the same reference numerals and signs are added, the same reference numerals and signs indicate the same components. Redundant explanation of the components is omitted. Illustrated specific examples are illustrations. Components other than those illustrated may be further included.

### First Embodiment

For example, a biological detection system 1 is a system having an overall configuration explained below.

### Overall Configuration Example

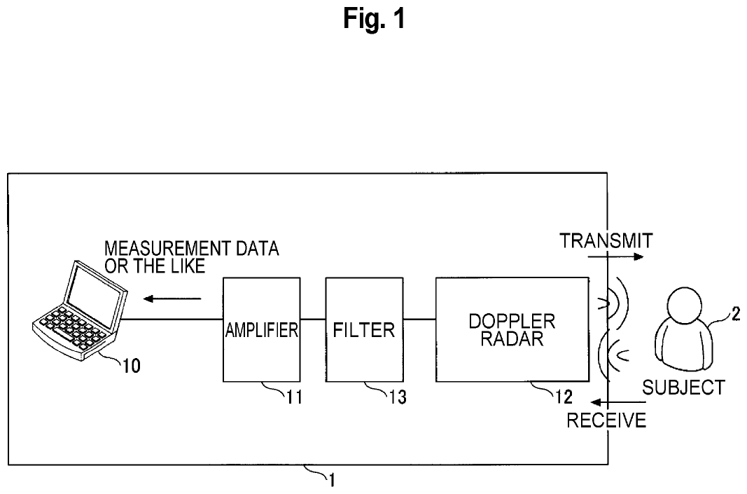

FIG. 1 is a diagram showing an overall configuration example in a first embodiment. For example, the biological detection system 1 has a configuration including a PC (Personal Computer, hereinafter referred to as “PC 10”), a Doppler radar 12, and a filter 13. Note that, as illustrated, the biological detection system 1 preferably includes an amplifier 11. In the following explanation, the illustrated overall configuration is explained as an example.

The PC 10 is an information processing device and is an example of a biological detection device. The PC 10 is connected to peripheral equipment such as the amplifier 11 via a network, a cable, or the like. Note that the amplifier 11 and the filter 13 may be components included in the PC 10. The amplifier 11, the filter 13, and the like may not be devices and may be configured by software or may be configured by both of hardware and software. In the following explanation, an illustrated example of the biological detection system 1 is explained.

The Doppler radar 12 is an example of a measurement device.

In this example, the PC 10 is connected to the amplifier 11. The amplifier 11 is connected to the filter 13. Further, the filter 13 is connected to the Doppler radar 12. The PC 10 acquires measurement data from the Doppler radar 12 through the amplifier 11 and the filter 13. That is, the measurement data is data of signals indicating motions of an organism such as respiration. Subsequently, the PC 10 analyzes body motions such as a heartbeat, respiration and a movement of the body of a subject 2 based on the acquired measurement data and measures a movement of a human body such as respiration.

The Doppler radar 12 acquires signals indicating motions such as a heartbeat and respiration (hereinafter referred to as “biological signals”), for example, in a principle explained below.

### Example of the Doppler Radar

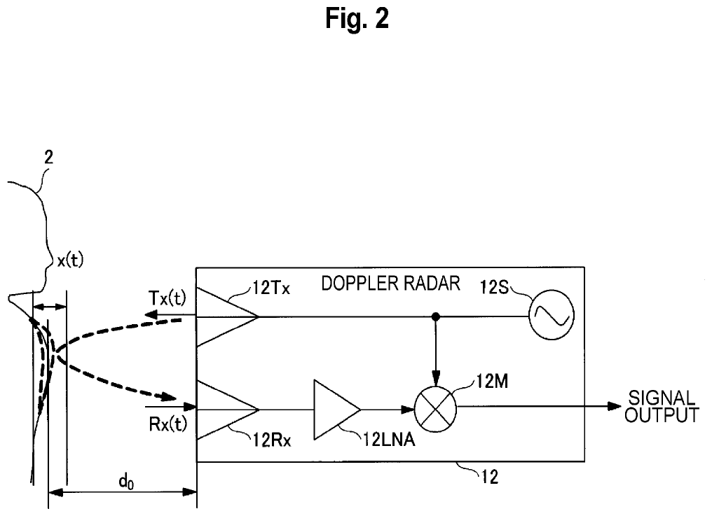

FIG. 2 is a diagram showing an example of the Doppler radar. For example, the Doppler radar 12 is a device having a configuration shown in FIG. 2. Specifically, the Doppler radar 12 includes a source 12S, a transmitter 12Tx, a receiver 12Rx, and a mixer 12M. The Doppler radar 12 includes an adjuster 12LNA such as an LNA (Low Noise Amplifier) that performs processing for, for example, reducing noise of data received by the receiver 12Rx.

The source 12S is a transmission source that generates a signal of a transmission wave transmitted by the transmitter 12Tx.

The transmitter 12Tx transmits a transmission wave to the subject 2. Note that a signal of the transmission wave can be indicated by a function Tx(t) relating to a time “t” and, for example, can be indicated by Expression (1) described below.

Math. 1

Tx(t)=cos(ω_{c}t) (Expression 1)

In Expression (1) described above, “ω_{c}” is an angular frequency of the transmission wave.

The subject 2, that is, a reflection surface of the transmitted signal is displaced by x(t) at the time “t”. In this example, the reflection surface is the chest wall of the subject 2. The displacement x(t) can be indicated by Expression (2) described below.

[Math. 2]

x(t)=m×cos(ωt) (Expression 2)

In Expression (2) described above, “m” is a constant indicating the amplitude of the displacement. In Expression (2) described above, “ω” is angular velocity shifted by a movement of the subject 2. Note that the variables common to Expression (1) described above are the same variables.

The receiver 12Rx receives a reflected wave that is a wave transmitted by the transmitter 12Tx and reflected by the subject 2. A signal of the reflected wave can be indicated by a function Rx(t) relating to the time t and, for example, can be indicated by Expression (3) described below.

$$[ Math . 3 ]$$

$$Rx ( t ) = cos ( {ω}_{c} t - 2 π \cdot \frac{2 ( {d}_{0} + x ( t ) )}{λ} ) \qquad ( Expression 3 )$$

In Expression (3) described above, “d₀” is the distance between the subject 2 and the Doppler radar 12. “λ” is a wavelength of the signal. The distance and the wavelength are described the same below.

The Doppler radar 12 mixes the function Tx(t) (Expression (1) described above) indicating the signal of the transmission wave and the function R(t) (Expression (3) described above) indicating the signal of the reception wave and generates a Doppler signal. Note that, in the case where the Doppler signal is indicated by a function B(t) relating to the time t, the Doppler signal can be indicated by Expression (4) described below.

$$[ Math . 4 ]$$

$$B ( t ) = cos ( θ + 2 π \cdot \frac{2 x ( t )}{λ} ) \qquad ( Expression 4 )$$

In the case where an angular frequency of the Doppler signal is represented as “ω_{d}”, the angular frequency ω_{d} of the Doppler signal can be indicated by Expression (5) described below.

$$[ Math . 5 ]$$

$${ω}_{d} = θ + 2 π \cdot \frac{2 x ( t )}{λ} \qquad ( Expression 5 )$$

A phase “θ” in Expression (4) described above and Expression (5) described above can be indicated by Expression (6) described below.

$$[ Math . 6 ]$$

$$θ = 2 π \cdot \frac{2 {d}_{0}}{λ} + {θ}_{0} \qquad ( Expression 6 )$$

In Expression (6) described above, “θ₀” is phase displacement on the chest wall of the subject 2, that is, the reflection surface.

Subsequently, the Doppler radar 12 outputs the position, the speed, and the like of the subject 2 based on a result of comparing the signal of the transmitted transmission wave and the signal of the received reception wave, that is, calculation results of the expressions described above.

For example, I data (in-phase data) and Q data (orthogonal phase data) can be generated from the reception wave. A distance that the chest wall of the subject 2 moved can be detected according to the I data and the Q data. It is possible to detect, based on phases indicated by the I data and the Q data, in which of the front and the rear the chest wall of the subject 2 moved. Therefore, an indicator such as a heartbeat and respiration can be detected from a movement of the chest wall deriving from a heartbeat using frequency changes of the transmission wave and the reception wave.

### Hardware Configuration Example of the Biological Detection Device

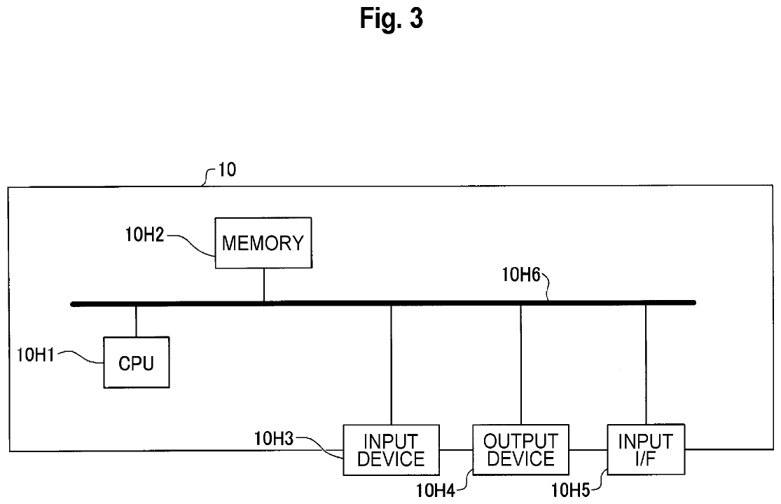

FIG. 3 is a diagram showing an example of the biological detection device. For example, the PC 10 includes a CPU (Central Processing Unit, hereinafter referred to as “CPU 10H1”), a memory 10H2, an input device 10H3, an output device 10H4, and an input I/F (Interface) (hereinafter referred to as “input I/F 10H5”). Note that the respective pieces of hardware included in the PC 10 are connected by a bus (hereinafter referred to as “bus 10H6”). Data and the like are mutually transmitted and received among the respective pieces of hardware through the bus 10H6.

The CPU 10H1 is a control device that controls the hardware included in the PC 10 and an arithmetic device that performs an arithmetic operation for realizing various kinds of processing.

The memory 10H2 is, for example, a main memory and an auxiliary memory. Specifically, the main memory is, for example, a memory. The auxiliary memory is, for example, a hard disk. The memory 10H2 stores data including intermediate data used by the PC 10, programs used for the various kinds of processing and the control, and the like.

The input device 10H3 is a device for inputting parameters and instructions necessary for calculation to the PC 10 according to operation of a user. Specifically, the input device 10H3 is, for example, a keyboard, a mouse, and a driver.

The output device 10H4 is a device for outputting various processing results and calculation results by the PC 10 to the user and the like. Specifically, the output device 10H4 is, for example, a display.

The input I/F 10H5 is an interface connected to an external device such as a measurement device to transmit and receive data and the like. For example, the input I/F 10H5 is a connector, an antenna, or the like. That is, the input I/F 10H5 transmits and receives data to and from the external device through a network, radio, or a cable.

Note that a hardware configuration is not limited to the illustrated configuration. For example, the PC 10 may further include an arithmetic device or a memory for performing processing in parallel, decentrally, or redundantly. The PC 10 may be an information processing system connected to other devices through a network or a cable in order to perform an arithmetic operation, control, and storage in parallel, decentrally, or redundantly. That is, the present invention may be realized by an information processing system including one or more information processing devices.

In this way, the PC 10 acquires a biological signal indicating a motion of an organism with the measurement device such as the Doppler radar 12. Note that the biological signal may be acquired at any time in real time or a device such as the Doppler radar may store biological signals for a certain period and, thereafter, the PC 10 may collectively acquire the biological signals. A recording medium or the like may be used for the acquisition. Further, the PC 10 may include the measurement device such as the Doppler radar 12 and the PC 10 may perform measurement with the measurement device such as the Doppler radar 12, generate a biological signal, and acquire the biological signal.

### Overall Processing Example

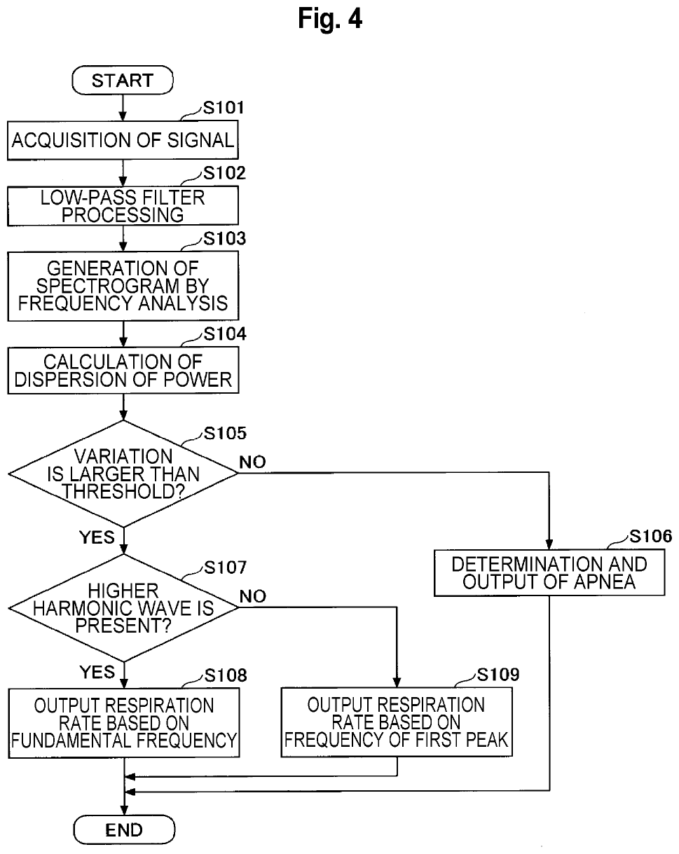

FIG. 4 is a diagram showing an overall processing example. For example, overall processing explained below is executed for each time window (for example, a value of approximately 30 seconds to 60 seconds is set beforehand).

### Example for Acquisition of Signal

In step S101, the PC 10 acquires a signal. For example, the PC 10 receives data from a measurement device such as a Doppler radar 12 and acquires a biological signal (hereinafter simply referred to as “signal”) indicated by a respiration component or the like.

### Example of Low-Pass Filter Processing

In step S102, the PC 10 may perform, on the signal, low-pass filter processing for attenuating a frequency component higher than a predetermined frequency component.

Specifically, the PC 10 performs the low-pass filter processing to attenuate a frequency component other than a frequency component corresponding to respiration.

The low-pass filter processing is preferably set to attenuate, for example, a frequency component higher than 3 Hz. In such setting, the PC 10 can attenuate, with the low-pass filter processing, a frequency component to be noise without attenuating the frequency component corresponding to the respiration.

Note that a frequency band set as a target of the low-pass filter processing may be set considering age, sex, a state, and the like of an organism. For example, in a state after vigorous exercise or in an excited state, both of a heart rate and a respiration rate have frequencies higher than frequencies in a quiet state. Therefore, in both of the heart rates and the respiration rate, frequency components included in signals are frequencies higher than the frequencies in the quiet states. On the other hand, in the quiet state, both of the heart rate and the respiration rate have low frequencies.

Therefore, for example, the frequency band set as the target of the low-pass filter processing may be dynamically changed according to the state or the like or the frequency band set as the target of the low-pass filter processing may be narrowed. Specifically, in a state in which the respiration rate is considered to have a high frequency component such as the state after a vigorous exercise, low-pass filter processing for attenuating a frequency component higher than 3.5 Hz is performed. On the other hand, in a state in which the respiration rate is considered to have a high frequency component such as the quiet state, low-pass filter processing for attenuating a frequency component higher than 1.4 Hz is performed.

In this way, the state or the like can be input or a value considering the state or the like may be set and the low-pass filter processing may be performed.

In the following explanation, a signal generated by performing the low-pass filter processing is referred to as “post-processing signal”.

### Example of Frequency Analysis

In step S103, the PC 10 performs a frequency analysis of the post-processing signal. For example, the frequency analysis is realized by FFT (Fast Fourier Transform) or the like. In this way, the PC 10 calculates a spectrum indicating energy for each frequency band. The PC 10 preferably performs normalization and indicates an analysis result as a spectrum. In the following explanation, the spectrum is indicated by a normalized value. A specific example of the analysis result is explained below.

In the analysis, a window function is preferably executed on the post-processing signal. Specifically, the window function is preferably a Hanning window function. Note that the window function may be another type. That is, the window function only has to be a function of cancelling temporal discontinuity and may be, for example, a rectangular window or a flat-top window.

In a state before the window function is processed, the post-processing signal often includes a discontinuous component in, for example, a part to be a boundary. Such a discontinuous component is often detected as a peak and often becomes a noise. Therefore, if the window function is used, it is possible to accurately analyze a frequency component.

### Calculation Example of Dispersion of Power

In step S104, the PC 10 calculates dispersion of power. First, energy is, for example, power generated in a predetermined frequency band among all frequency bands indicated by a spectrogram. In the following explanation, an example is explained in which the energy is power. That is, the energy is calculated by the PC 10 from a change in the voltage (or the electric current) on a lead wire after the energy of an electromagnetic wave is guided to the lead wire by an antenna. In this way, the energy only has to be a value indicating the energy at each of times and frequencies. An acquiring method and a calculating method for the energy do not matter.

The predetermined frequency band is preferably 0.07 Hz to 0.58 Hz (that is, targets a frequency of approximately 4 to 35 bpm (Beats Per Minute)). That is, the power is preferably narrowed to power generated in a frequency band in which a respiration rate is highly likely to occur. Therefore, the predetermined frequency band may be set to a frequency band, a range of which is more limited or expanded than or different from 0.07 Hz to 0.58 Hz, according to a state or the like of a subject.

In the following explanation, an example is explained in which a frequency band of 0.07 Hz to 0.58 Hz is set as the “predetermined frequency band”.

Subsequently, dispersion of power (hereinafter simply referred to as “dispersion”) is calculated. That is, a degree of “variation” in energy is converted into a numerical value by the dispersion. Note that the variation only has to be a value indicating a variation degree such as dispersion or standard deviation. In the following explanation, an example is explained in which the variation is indicated by the dispersion.

### Example of Determination about whether Variation is Larger than Threshold

In step S105, the PC 10 determines whether variation is larger than a threshold. That is, in this example, it is determined whether dispersion is a large value or a small value.

The threshold is a value serving as a reference of determination set beforehand. For example, the threshold is a value calculated by averaging dispersion from a preceding fixed time to a present time. In this way, the threshold is calculated by, for example, multiple time windows.

Subsequently, if it is determined that the dispersion is larger than the threshold (YES in step S105), the PC 10 proceeds to step S107. On the other hand, if it is determined that the dispersion is not larger than the threshold (NO in step S105), the PC 10 proceeds to step S106.

### Example in which Apnea is Determined and Output

In step S106, the PC 10 determines and outputs apnea. For example, the PC 10 determines that the subject is in a state of apnea and outputs “0” as a respiration rate. Note that the output in the case in which the apnea is determined may be a value other than “0” or an output such as a message.

If the apnea is determined according to whether the value of the dispersion is larger than the threshold as in step S104 to step S106, it is possible to accurately determine the apnea.

### Example of Determination about whether Higher Harmonic Wave is Present

In step S107, the PC 10 determines whether a higher harmonic wave is present.

Presence or absence of a higher harmonic wave is determined based on a peak where power is maximum (hereinafter referred to as “first peak”) in the predetermined frequency band. Therefore, fist, the PC 10 specifies the first peak. Subsequently, the PC 10 determines whether a predetermined number or more of peaks where power equal to or more than a predetermined ratio to the first peak is present (hereinafter referred to as “second peaks”) occur.

The predetermined ratio is a value serving as a reference for determining whether a peak is the second peak. Note that the predetermined ratio is set beforehand. Specifically, the predetermined ratio is preferably set to approximately 20% to 40%. More preferably, the predetermined ratio is set to approximately 30%. In the following explanation, an example is explained in which the predetermined ratio is set to “30%”.

That is, if a peak of large power having power equal to or more than 30% of the first peak is present in the predetermined frequency band, it is determined that the peak is the second peak. The number of second peaks is counted and it is determined whether a predetermined number or more of second peaks occur.

The predetermined number is set beforehand. Specifically, the predetermined number is set to, for example, three. In the following explanation, an example is explained in which the predetermined number is set to “three”. Therefore, in the case where three or more second peaks occur, that is, in the case where the second peaks occur by a certain degree of a number, processing for determining presence or absence of a higher harmonic wave explained below is continuously performed.

Subsequently, the PC 10 selects three peaks from a low frequency among the second peaks. In the following explanation, the peaks selected in this way are referred to as “candidate peaks”. The PC 10 determines whether a candidate peak is a higher harmonic wave integer times as large as the fundamental wave. In this way, it is determined whether the candidate peak is a higher harmonic wave. If the candidate peak is a higher harmonic wave, that is, the candidate peak corresponds to integer times of the fundamental wave, it is determined that a higher harmonic wave is present (YES in step S107). Note that presence or absence of a higher harmonic wave may be determined by a method other than the above.

If it is determined that a higher harmonic wave is present (YES in step S107), the PC 10 proceeds to step S108. On the other hand, if it is determined that a higher harmonic wave is absent (NO in step S107), the PC 10 proceeds to step S109.

### Example in which Respiration Rate Based on Fundamental Frequency is Output

In step S108, the PC 10 outputs a respiration rate based on the fundamental frequency.

The fundamental frequency is a frequency of a fundamental wave. For example, the fundamental frequency is a frequency of a peak having the lowest frequency among peaks determined as higher harmonic waves. If it is determined in this way that a higher harmonic wave is present, the PC 10 outputs the specified fundamental frequency as a respiration rate.

### Example in which Respiration Rate Based on Frequency of First Peak is Output

In step S109, the PC 10 outputs a respiration rate based on a frequency of the first peak. That is, the PC 10 outputs a frequency having the largest power in the predetermined frequency band as a respiration rate.

If the respiration rate is specified as explained above, it is possible to accurately measure and output respiration. In particular, it is possible to accurately measure respiration even if a state of apnea is present.

### Second Embodiment

A second embodiment is different from the first embodiment in overall processing. In the following explanation, differences from the first embodiment are mainly explained and redundant explanation is omitted.

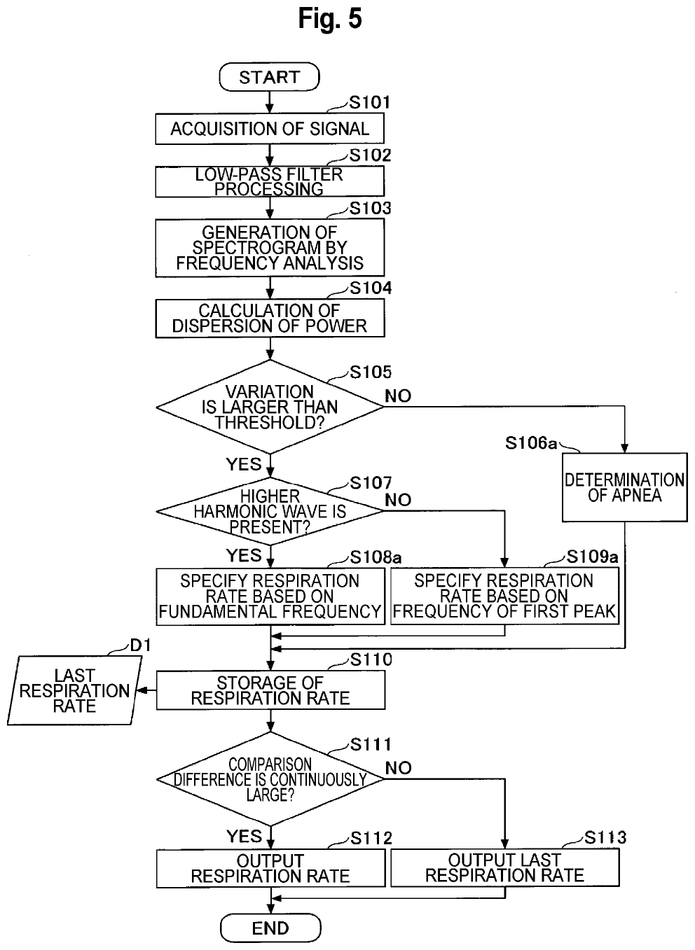

FIG. 5 is a diagram showing an overall processing example in the second embodiment. Compared with the first embodiment, the second embodiment is different in that processing in step S110 and subsequent steps is added.

First, in the second embodiment, a respiration rate is specified in step S106a, step S108a, or step S109a. That is, unlike the first embodiment, the specified respiration rate is not immediately output and the processing in step S110 and subsequent steps is performed based on the specified respiration rate.

### Example in which Respiration Rate is Stored

In step S110, the PC 10 stores the specified respiration rate. In the following explanation, a respiration rate measured before stored in this way is referred to as “last respiration rate D1”. Therefore, in processing in step S111 and subsequent steps, the last respiration rate D1 stored in step S110 before is read out and used.

### Example in which it is Determined whether Comparison Difference is Continuously Large

In step S111, the PC 10 determines whether a comparison difference is continuously large. For example, specifically, the PC 10 compares a respiration rate specified this time (hereinafter simply referred to as “respiration rate”) and the last respiration rate D1 and determines, based on a comparison result, whether the comparison difference is continuously large.

For example, if the difference between the respiration rate and the last respiration rate D1 is continuously larger than a threshold for comparison, it is determined that the comparison difference is continuously large.

First, the threshold for comparison is preferably set to, for example, approximately 0.08 Hz (approximately 5 bpm).

Subsequently, the PC 10 preferably counts the number of times the difference between the respiration rate and the last respiration rate D1 is continuously larger than the threshold for comparison and determines, based on the counted number of times, whether the comparison difference is continuously large. According to whether the number of times is continuously larger than a number of times threshold, the PC 10 determines whether the comparison difference is continuously large. Specifically, the number of times threshold is set to approximately two beforehand.

Therefore, if the difference between the respiration rate and the last respiration rate D1 is larger than the threshold for comparison approximately once to twice, the PC 10 determines that the comparison difference is continuously large (YES in step S111).

Subsequently, if it is determined that the comparison difference is continuously large (YES in step S111), the PC 10 proceeds to step S112. On the other hand, if it is determined that the comparison difference is not continuously large (NO in step S111), the PC 10 proceeds to step S113.

### Example in which respiration rate is output

In step S112, the PC 10 outputs the respiration rate.

(Example in which last respiration rate is output)

In step S113, the PC 10 outputs the last respiration rate.

If the comparison difference is continuously large, possibility of misdetection is high. Therefore, the PC 10 outputs the last respiration rate (step S113). On the other hand, if the comparison difference is not continuously large, the PC 10 outputs the respiration rate measured this time (step S112).

For example, the PC 10 may output the respiration rate measured this time based on one comparison result. That is, the PC 10 outputs the respiration rate measured this time according to one comparison result indicating that the comparison difference is not large. On the other hand, the PC 10 outputs the last respiration rate D1 according to one comparison result indicating that the comparison difference is large. In this way, the output may be switched according to only the one comparison result.

On the other hand, a plurality of comparison results may be used. For example, the PC 10 outputs the last respiration rate D1 in the case of a comparison result indicating that the comparison difference is continuously large twice.

Besides, the PC 10 outputs the respiration rate measured this time in the case of one comparison result indicating that the comparison difference is not large next to a comparison result indicating that the comparison difference is large approximately once or twice. In this way, content to be output may be selected using a continuous plurality of comparison results or the like.

Note that it may be optionally set whether “continuously” is set as a requirement. A degree of the number of times such as “once or twice” may be optionally set.

If the last respiration rate is used in this way, it is possible to more accurately measure respiration.

The determination of the apnea by the dispersion is preferably performed at timing earlier than the processing such as the comparison as in step S105. At such timing, it is possible to accurately determine the apnea.

### Experiment Results

First, two examples of results obtained by performing experiments on normal subjects, that is, in a state in which apnea is absent (referred to as first experiment and second experiment) are explained. Note that subjects are different in the two examples explained below.

In the following explanation, a true value is indicated as “Ground-truth of respiration rate” or “GT” in the experiments.

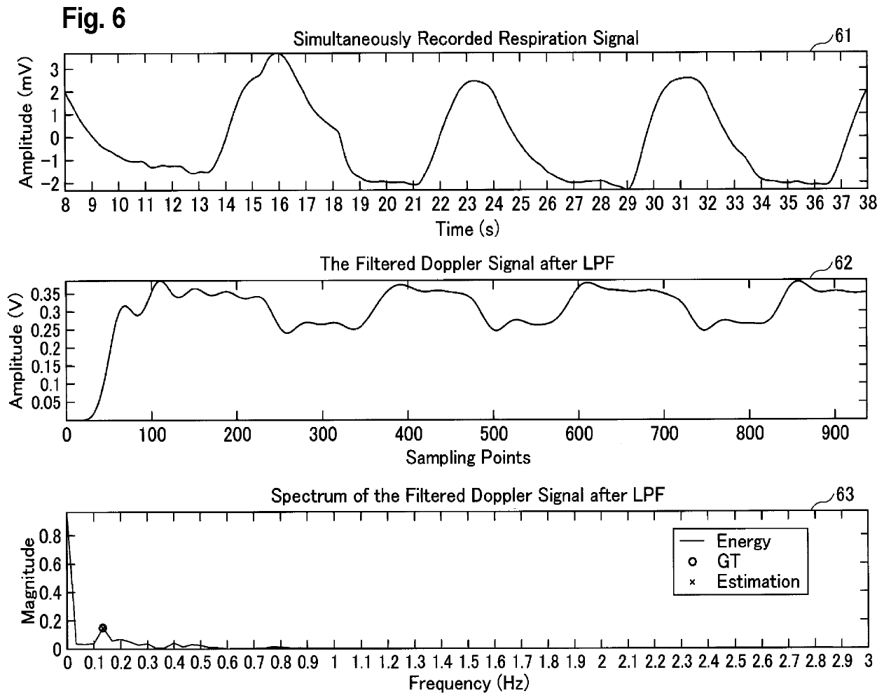

FIG. 6 is a diagram showing a first experiment result. A result of acquiring a signal indicated by the waveform 61 and subjecting the signal to low-pass filter processing is a waveform 62. A spectrum is indicated by a waveform 63. An output respiration rate is indicated by an “X” mark in the waveform 63.

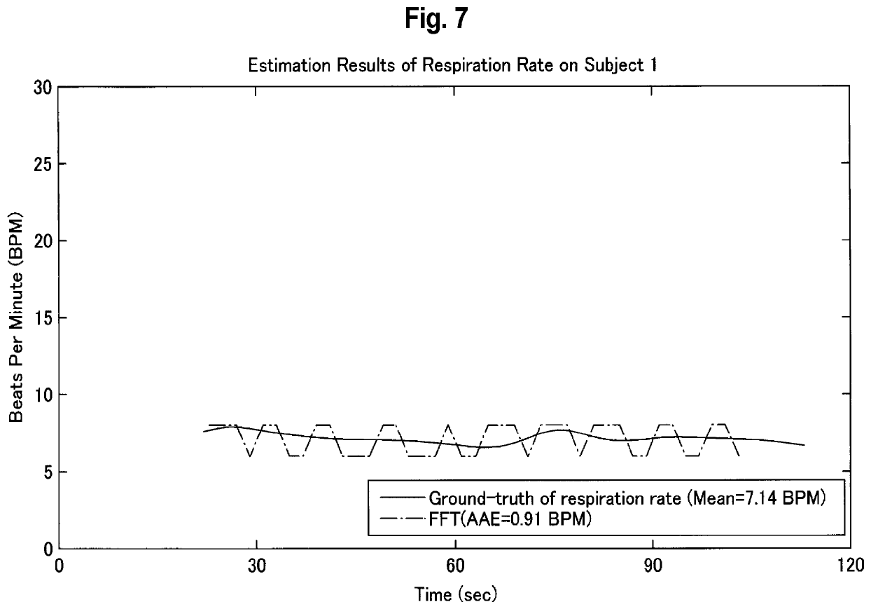

FIG. 7 is a diagram showing an evaluation result of comparison with the true value in the first experiment. In the figure, an output result is indicated by “FFT”. Comparing the output shown in FIG. 5 with the true value, an error (in the figure, indicated by “AAE”) was approximately “0.91 bpm”.

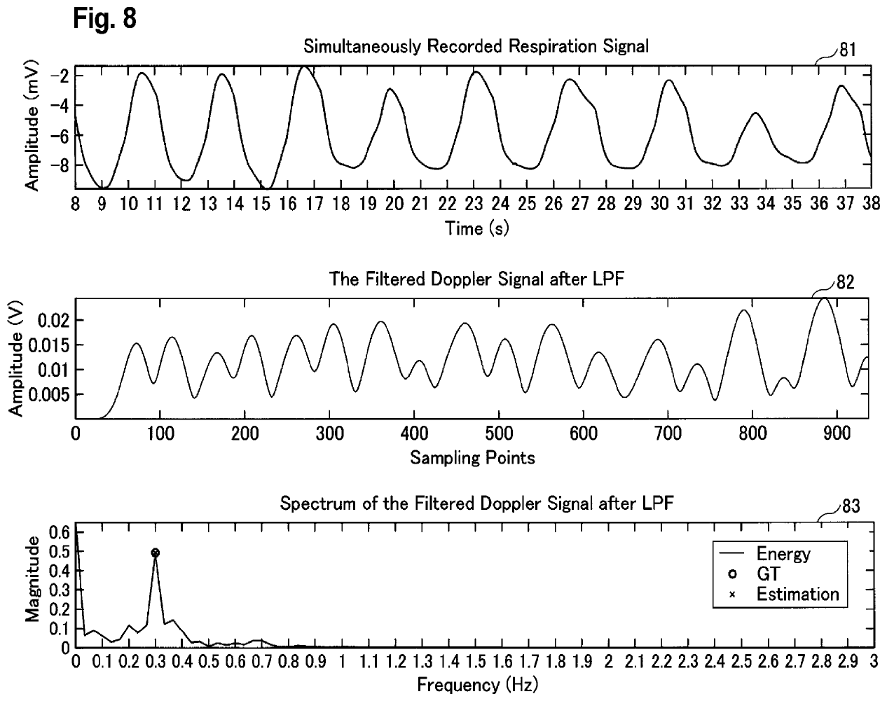

FIG. 8 is a diagram showing a second experiment result. Waveforms 81 and 82 and a waveform 83 indicate the same data as the data of the first experiment.

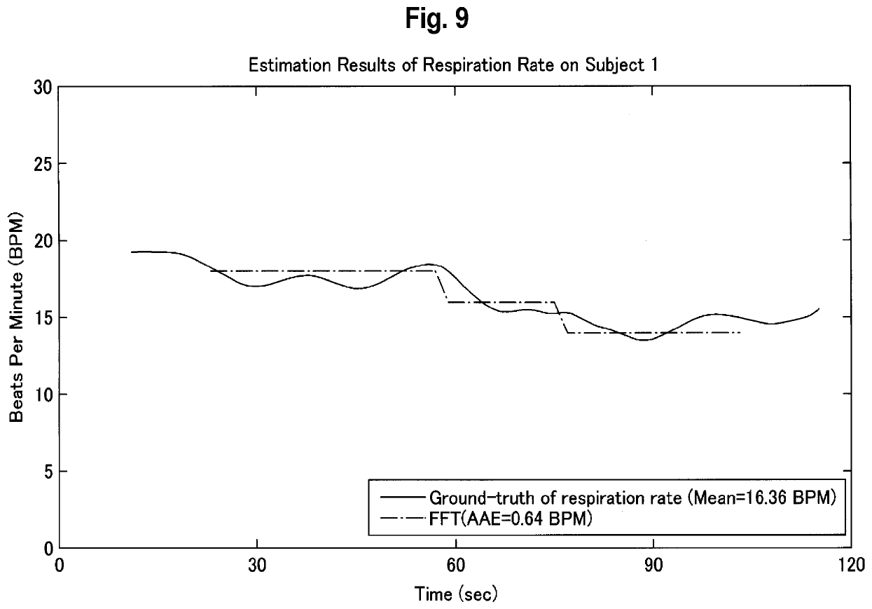

FIG. 9 is a diagram showing an evaluation result of comparison with the true value in the second experiment. In the figure, an output result is indicated by “FFT”. Comparing the output shown in FIG. 7 with the true value, an error (in the figure, indicated by “AAE”) was approximately “0.64 bpm”.

Note that, in the first experiment and the second experiment, the subjects were in a state of lying on their back in a bedroom, that is, in a quiet state. A time window width is 30 seconds.

As explained above, a respiration rate was successfully accurately measured.

Subsequently, an experiment result in the case in which apnea is present, that is, an abnormality occurs in the subjects is explained.

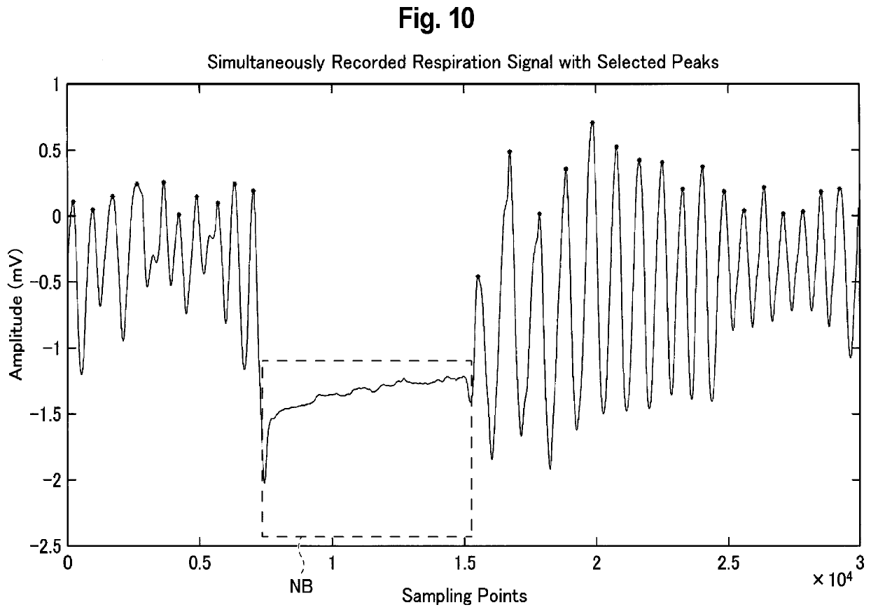

FIG. 10 is a diagram showing an occurrence example of apnea. The figure is a measurement result by a respiration belt. A state indicated by an “apnea state NB” in this experiment is an example in which apnea occurs. On the other hand, circle marks in the figure are examples of “peaks”. A result of calculating dispersion in this example is explained. Note that a time window width is 20 seconds. In the following explanation, explanation is divided into a case in which “one” time window is set (hereinafter referred to as “single time window”) and a case in which “three” time windows are set (hereinafter referred to as “three time windows”).

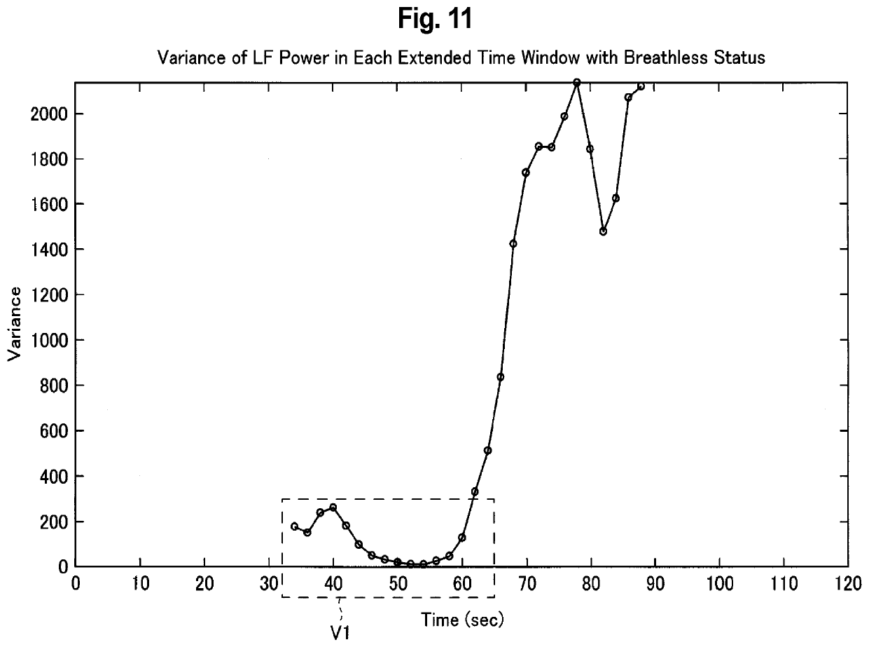

FIG. 11 is a diagram showing a calculation result of dispersion by the single time window. In the apnea state NB, the dispersion is a small value like a “first dispersion value V1” in the figure.

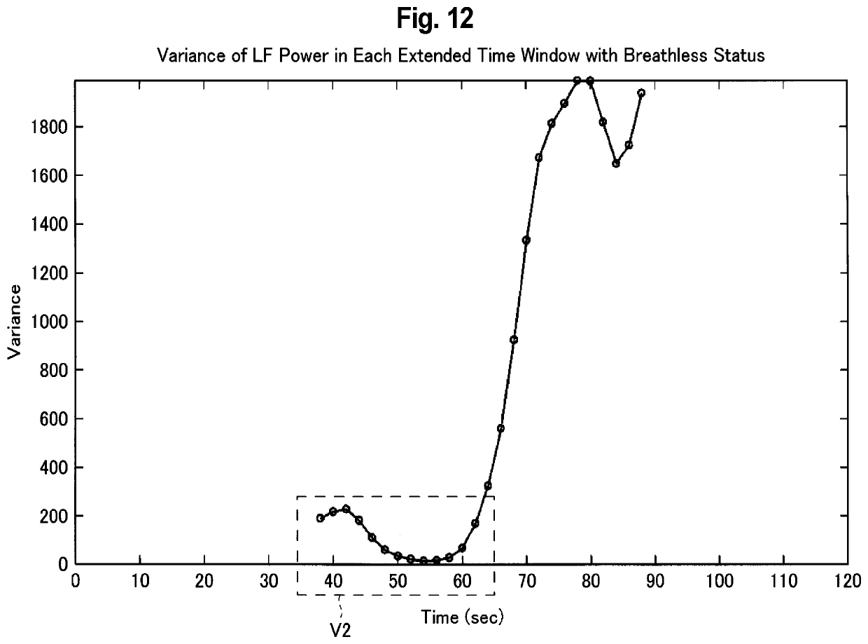

FIG. 12 is a diagram showing a calculation result of dispersion by the three time windows. In the apnea state NB, dispersion is a small value like a “second dispersion value V2” in the figure.

Therefore, if a case in which a value of dispersion is small is detected, it is possible to accurately measure the apnea state NB.

In the following explanation, an experiment result obtained by setting states of the first dispersion value V1 and the second dispersion value V2 as the apnea state NB and specifying a respiration rate as “0” and outputting the respiration rate is explained below.

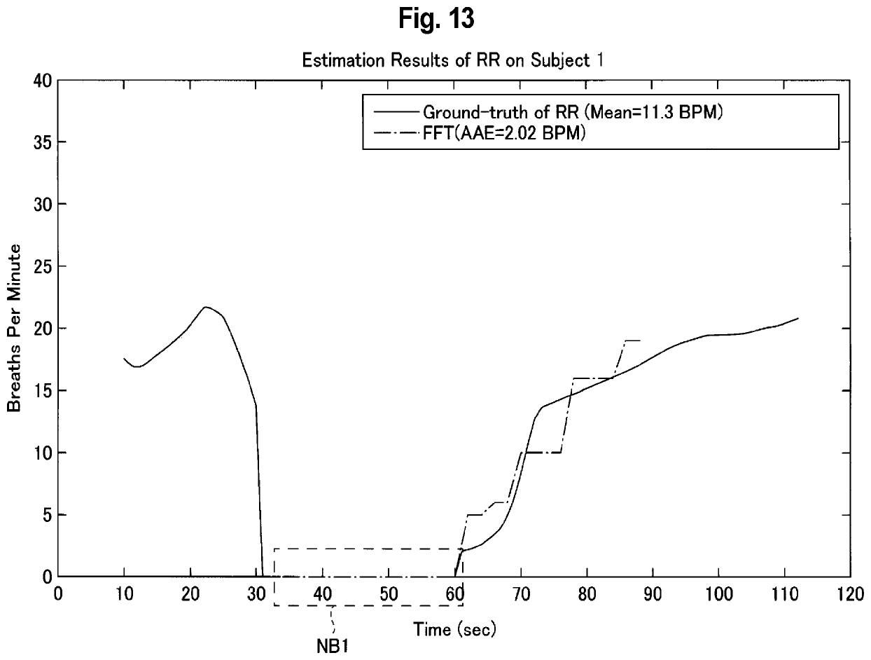

FIG. 13 is a diagram showing an evaluation result of comparison with the true value in the experiment performed using the single time window. In the figure, a first apnea determination result NB1 is a result of determining apnea.

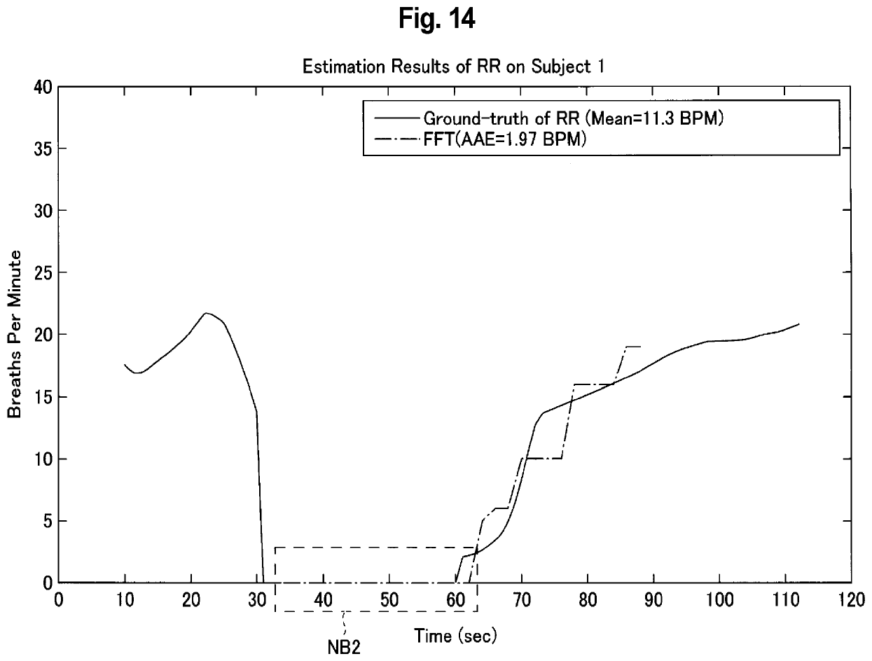

FIG. 14 is a diagram showing an evaluation result of comparison with the true value in the experiment performed using the three time windows. In the figure, a second apnea determination result NB2 is a result of determining apnea.

As it is seen when compared with the true value, the first apnea determination result NB1 and the second apnea determination result NB2 can be accurately determined. After the first apnea determination result NB1 and the second apnea determination result NB2, the respiration rate correctly recovers like the true value and the respiration rate accurately follows the true value.

As explained above, the number of time windows is preferably set to approximately three or less. If the number of time windows is more than three (is, for example, five), the time windows more often exceed a time of apnea. Therefore, respiration can be accurately measured if the number of time windows is approximately three or less.

### Comparative Experiment Results

An effect of considering the higher harmonic wave as in step S107 to step S109 in FIG. 4 and FIG. 5 is indicated by experiment results explained below.

First, experiment results in the case in which the higher harmonic wave is considered (referred to as third experiment and fourth experiment) are explained below. Note that subjects are different in two examples explained below. Note that, in the third experiment and the fourth experiment, the subjects were in a state of lying on their back in a bedroom, that is, a quiet state.

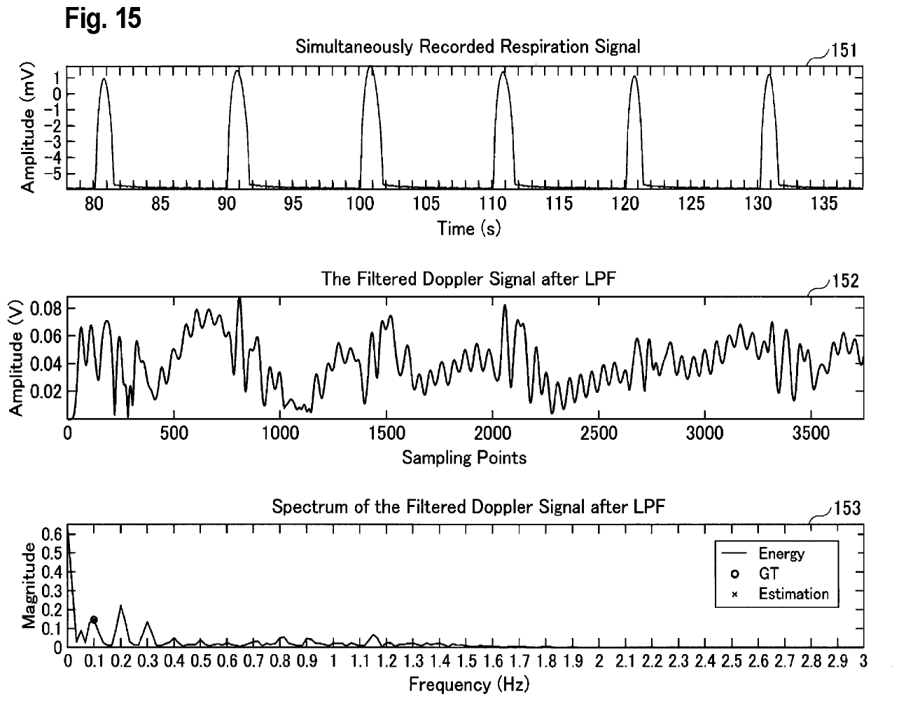

FIG. 15 is a diagram showing a third experiment result. Waveforms 151, 152, and 153 indicate the same data as the data of the first experiment.

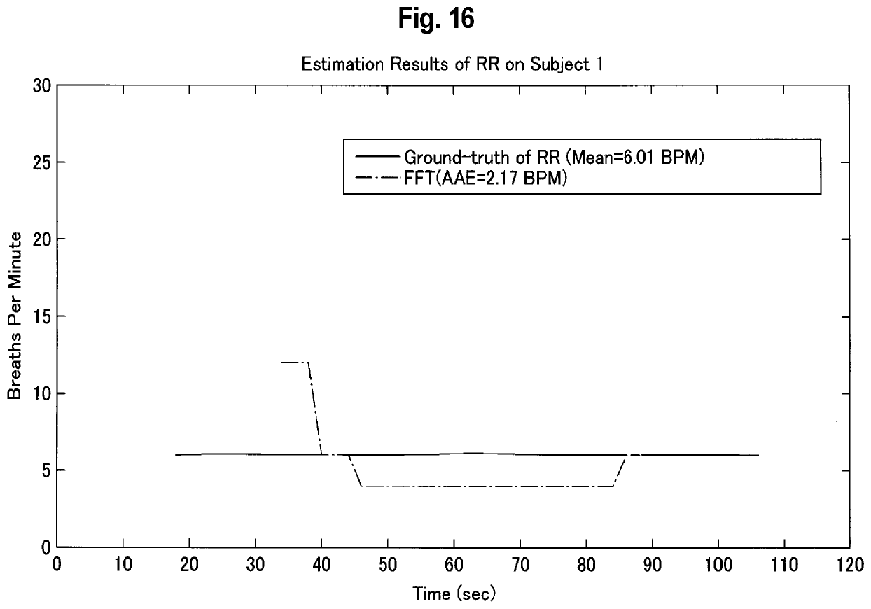

FIG. 16 is a diagram showing an evaluation result of comparison with the true value in the third experiment. In the figure, an output result is indicated by “FFT”. Comparing the output shown in FIG. 7 with the true value, an error (in the figure, indicated as “AAE”) was approximately “2.17 bpm”.

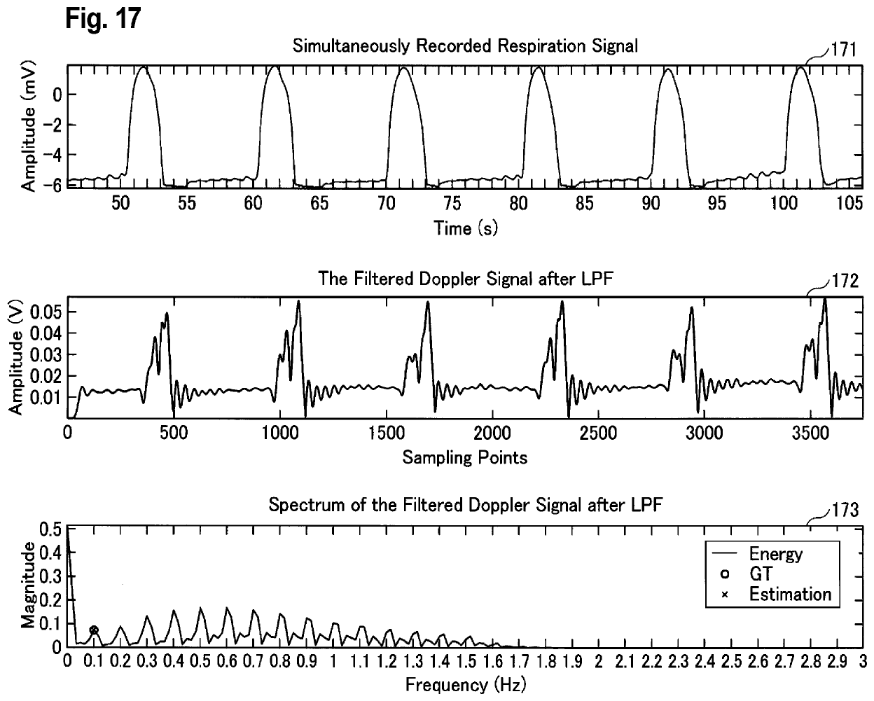

FIG. 17 is a diagram showing a fourth experiment result. Waveforms 171, 172, and 173 indicate the same data as the data of the first experiment.

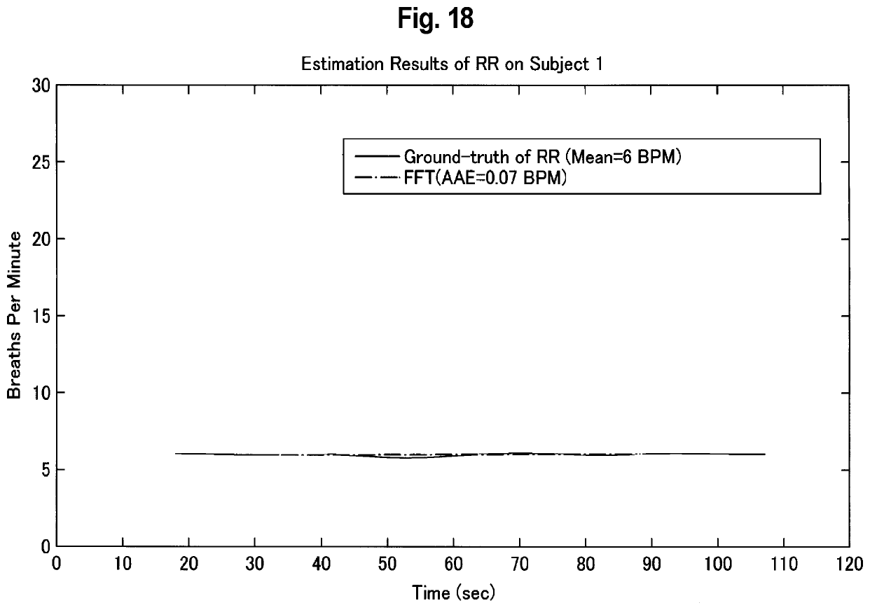

FIG. 18 is a diagram showing an evaluation result of comparison with the true value in the fourth experiment. In the figure, an output result is indicated by “FFT”. Comparing the output shown in FIG. 7 with the true value, an error (indicated as “AAE” in the figure) was approximately “0.07 bpm”.

As explained above, by corresponding to the higher harmonic wave, respiration can be accurately measured.

In the following explanation, for comparison, results of measurement by a method of not using the higher harmonic wave at the same time (hereinafter referred to as “comparative examples”) are explained. That is, in a first comparative example and a second comparative example explained below, a state and the like of subjects are the same as those in the third experiment and the fourth experiment.

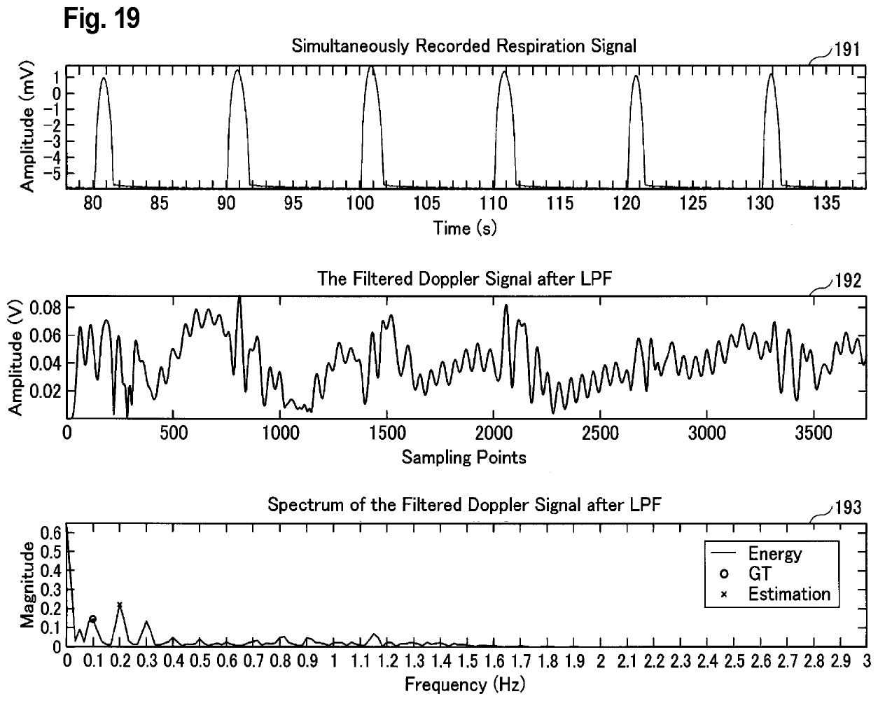

FIG. 19 is a diagram showing the first comparative example. Waveforms 191, 192, and 193 indicate the same data as the data of the first experiment. As indicated by the waveform 193, a result in which the true value (a value indicated by a “◯” mark”) and an output respiration rate (a value indicated by a “X” mark) greatly deviated was obtained.

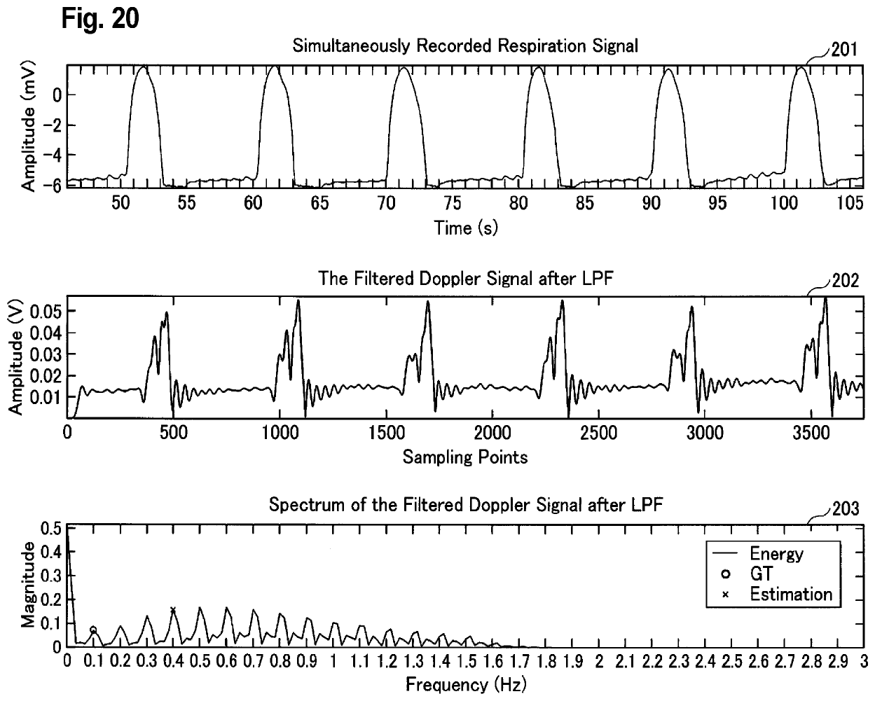

FIG. 20 is a diagram showing the second comparative example. Waveforms 201, 202, and 203 indicate the same data as the data of the first experiment. As indicated by the waveform 203, a result in which the true value (“a value indicated by a “◯” mark) and an output respiration rate (a value indicated by a “X” mark) greatly deviated was obtained.

### Functional Configuration Example

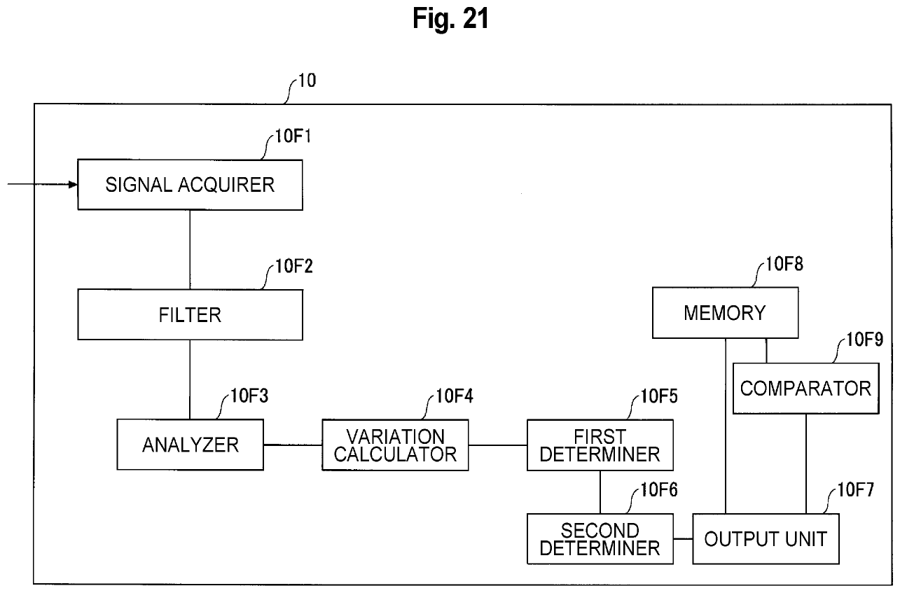

FIG. 21 is a diagram showing a functional configuration example. For example, the biological detection device has a functional configuration including a signal acquirer 10F1, a filter 10F2, an analyzer 10F3, a dispersion calculator 10F4, a first determiner 10F5, a second determiner 10F6, and an output unit 10F7. The biological detection device preferably has the functional configuration further including a memory 10F8 and a comparator 10F9 as illustrated. In the following explanation, the illustrated functional configuration is explained as an example.

The signal acquirer 10F1 performs a signal acquisition procedure for acquiring a signal including respiration. For example, the signal acquirer 10F1 is realized by the Doppler radar 12, an input I/F 10H5, or the like.

The filter 10F2 performs a filter procedure for attenuating a frequency component other than a frequency component of the respiration included in the signal acquired by the signal acquirer 10F1 and generating a post-processing signal. For example, the filter 10F2 is realized by a CPU 10H1, a filter 13, or the like.

The analyzer 10F3 performs an analysis procedure for analyzing the post-processing signal generated by the filter 10F2 and generating a spectrogram. For example, the analyzer 10F3 is realized by the CPU 10H1 or the like.

The variation calculator 10F4 performs a variation calculation procedure for calculating variation of energy in the predetermined frequency band in energy of a frequency component indicated by the spectrogram generated by the analyzer 10F3. For example, the variation calculator 10F4 is realized by the CPU 10H1 or the like.

The first determiner 10F5 performs a first determination procedure for determining whether the variation calculated by the variation calculator 10F4 is larger than a threshold. For example, the first determiner 10F5 is realized by the CPU 10H1 or the like.

The second determiner 10F6 performs a second determination procedure for, if it is determined by the first determiner that the variation is not larger than the threshold, determining whether the higher harmonic wave with respect to the second peak is present in the predetermined frequency band. For example, the second determiner 10F6 is realized by the CPU 10H1 or the like.

The output unit 10F7 performs an output procedure for, if it is determined by the second determiner 10F6 that the higher harmonic wave is absent, outputting a respiration rate based on the frequency of the first peak. On the other hand, the output unit 10F7 performs an output procedure for, if it is determined by the second determiner 10F6 that the higher harmonic wave is present, outputting a respiration rate based on the fundamental frequency of the higher harmonic wave. For example, the output unit 10F7 is realized by an output device 10H4 or the like.

The memory 10F8 performs a storage procedure for storing the last respiration rate. For example, the memory 10F8 is realized by a memory 10H2 or the like.

The comparator 10F9 performs a comparison procedure for comparing the respiration rate and the last respiration rate. For example, the comparator 10F9 is realized by the CPU 10H1 or the like.

### Example of IQ Data Measured by Doppler Radar

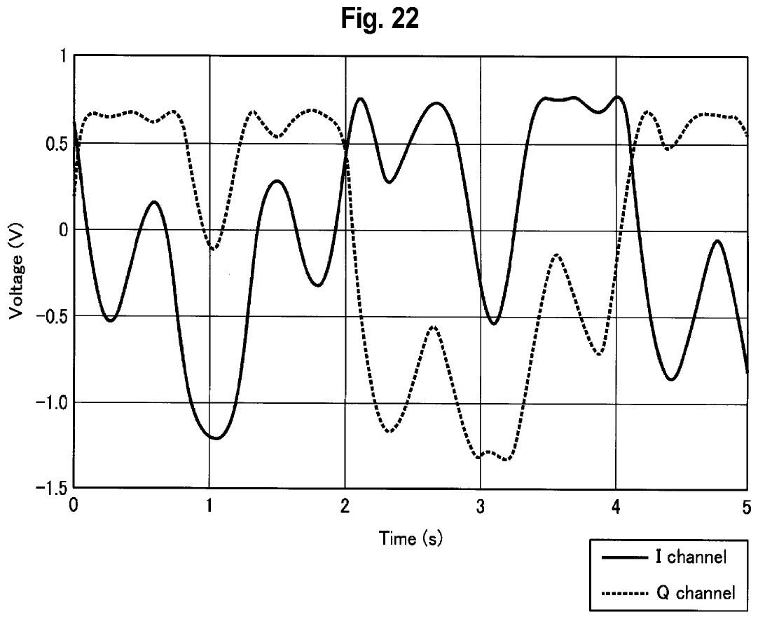

FIG. 22 is a diagram showing an example of IQ data measured by a Doppler radar. For example, the Doppler radar 12 outputs an illustrated signal. If arctan (Q/I) is calculated, a biological signal is obtained.

The Doppler radar 12 can measure, by irradiating a moving target object with a radio wave, a movement of the target object can be measured based on a Doppler effect in which a frequency of a reflected wave changes. A configuration that can measure a movement of a subject in a noncontact manner in this way is desirable.

### Modifications

Note that the organism is not limited to a human and may be an animal or the like.

The biological detection device and the biological detection system may be configured to use AI (Artificial Intelligence). A setting value such as a threshold may be learned by machine learning or the like and set.

The biological detection device and the biological detection system may perform deep learning with a time domain signal or a frequency domain signal set as a target of learning. The biological detection device and the biological detection system may determine various kinds of setting and determination based on a learned model.

The learned model is used as a part of software in AI. Therefore, the learned model is a program. Therefore, the learned model may be distributed or executed via, for example, a recording medium or a network.

The learned model includes a network structure such as a CNN (Convolution Neural Network) or an RNN (Recurrent Neural Network). The learned model may be realized by the Cloud or the like that can be used through a network or the like.

As explained above, functional components may not include both of a component for “learning processing” and a component for “execution processing”. For example, in a stage where the “learning processing” is performed, the functional components may not include the component for “execution processing”. Similarly, in a stage where the “execution processing” is performed, the functional components may not include the component for “learning processing”. In this way, the functional components can be divided for the stages of “learning” and “execution” and formed as components excluding components different from the components for the processing to be performed. Note that, for example, after the “learning processing” or the “learning processing”, various settings in the network structure may be adjusted by the user.

Note that the biological detection device or the biological detection system may calculate an indicator concerning the organism other than the respiration rate. For example, the indicator is a value indicating biological information of a target organism. Specifically, the indicator is a value calculated by analyzing a biological signal and is a pulse rate, a heart rate, a respiration rate, a blood pressure, a PTT (pulse transit time), a systolic blood pressure, an RRI (R-R interval), a QRS interval, a QT interval, a combination of the foregoing, or the like. Note that the indicator may be biological information other than the above. In order to calculate the indicator, the biological detection device or the biological detection system may perform the procedures from the acquisition of the biological signal.

The apnea is not limited to a case in which the respiration rate is completely “0”. The apnea only has to be a value indicating a case in which the organism has an abnormal respiration rate (in particular, a case in which the respiration rate is abnormally low).

Therefore, the respiration rate regarded as the apnea may be individually defined depending on a person, a state, or the like. For example, the apnea may be a case of a low respiration rate such as 8 bpm or less.

In this way, the state of the apnea is a dangerous state for the organism or a state in which health is highly likely to be ruined. Therefore, if the state of the apnea is accurately determined and output, it is possible to quickly cope with, for example, a case in which the organism is in danger.

### Other Embodiments

For example, the transmitter, the receiver, and the information processing devices may be pluralities of devices. That is, the processing and the control may be performed virtually, in parallel, decentrally, or redundantly. On the other hand, hardware may be integrated with or may function as the transmitter, the receiver, and the information processing device.

All or a part of the kinds of processing according to the present invention may be described in a low-level language such as assembler or a high-level language such as an object-oriented language and realized by a program for causing a computer to execute the biological detection method. That is, the program is a computer program for causing a computer such as an information processing device or a biological detection system to executes the kinds of processing.

Therefore, when the kinds of processing are executed based on the program, an arithmetic device and a control device included in the computer perform an arithmetic operation and control based on the program in order to execute the kinds of processing. A memory included in the computer stores data used for the processing based on the program in order to execute the kinds of processing.

The program can be recorded in a computer-readable recording medium and distributed. Note that the recording medium is a medium such as a magnetic tape, a flash memory, an optical disk, a magneto-optical disk, or a magnetic disk. Further, the program can be distributed through an electric communication line.

The preferred embodiments and the like are explained above. However, without being limited to the embodiments and the like explained above, various modifications and substitutions can be applied to the embodiments and the like explained above without departing from the scope described in the claims.

This international application claims the priority based on Japanese Patent Application No. 2020-145624 filed on Aug. 31, 2020, the entire content of Japanese Patent Application No. 2020-145624 being incorporated in this international application.

### REFERENCE SIGNS LIST

1 Biological detection system
2 Subject
10F1 Signal acquirer
10F2 Filter
10F3 Analyzer
10F4 Dispersion calculator
10F5 First determiner
10F6 Second determiner
10F7 Output unit
10F8 Memory
10F9 Comparator
11 Amplifier
12 Doppler radar
13 Filter
D1 Last respiration rate
NB Apnea state
V1 First dispersion value
V2 Second dispersion value

1. A biological detection device comprising:
a signal acquirer that acquires a signal including a respiration component;
a filter that attenuates a frequency component other than the respiration component included in the signal and generates a post-processing signal;
an analyzer that analyzes the post-processing signal to generate a spectrogram;
a variation calculator that calculates variation of energy in a predetermined frequency band in energy of the frequency component indicated by the spectrogram;
a first determiner that determines whether the variation is larger than a threshold;
a second determiner that, if it is determined by the first determiner that the variation is not larger than the threshold, determines whether a higher harmonic wave with respect to a second peak having energy equal to or more than a predetermined ratio to a first peak where the energy is maximum is present in the predetermined frequency band; and
an output unit that outputs a respiration rate based on a frequency of the first peak if it is determined by the second determiner that the higher harmonic wave is absent and outputs a respiration rate based on a fundamental frequency of the higher harmonic wave if it is determined by the second determiner that the higher harmonic wave is present.

2. The biological detection device according to claim 1, wherein the analyzer executes a window function on the post-processing signal.

3. The biological detection device according to claim 1, wherein the predetermined frequency band is 0.07 Hz to 0.58 Hz.

4. The biological detection device according to claim 1, wherein, if it is determined by the first determiner that the variation is larger than the threshold, the output unit outputs determination of apnea.

5. The biological detection device according to claim 1, further comprising:
a memory that stores a last respiration rate output earlier than the respiration rate; and
a comparator that compares the respiration rate output by the output unit and the last respiration rate, wherein
the output unit outputs the last respiration rate or outputs the respiration rate according to a comparison result by the comparator.

6. The biological detection device according to claim 5, wherein the comparator compares whether a difference between the respiration rate and the last respiration rate is larger than a threshold for comparison.

7. The biological detection device according to claim 6, wherein
the comparator counts a number of times the difference between the respiration rate and the last respiration rate is continuously larger than the threshold for comparison, and
the output unit outputs the last respiration rate or outputs the respiration rate according to a comparison result by the number of times.

8. The biological detection device according to claim 1, wherein the signal acquirer acquires the signal with a Doppler radar.

9. The biological detection device according to claim 1, wherein the variation is dispersion.

10. A biological detection method performed by a biological detection device, the biological detection method comprising:
a signal acquisition procedure in which the biological detection device acquires a signal including a respiration component;
a filter procedure in which the biological detection device attenuates a frequency component other than the respiration component included in the signal and generates a post-processing signal;
an analysis procedure in which the biological detection device analyzes the post-processing signal to generate a spectrogram;
a dispersion calculation procedure in which the biological detection device calculates variation of energy in a predetermined frequency band in energy of each frequency component indicated by the spectrogram;
a first determination procedure in which the biological detection device determines whether the variation is larger than a threshold;
a second determination procedure in which, if it is determined in the first determination procedure that the variation is not larger than the threshold, the biological detection device determines whether a higher harmonic wave with respect to a second peak having energy equal to or more than a predetermined ratio to a first peak where the energy is maximum is present in the predetermined frequency band; and
an output procedure in which the biological detection device outputs a respiration rate based on a frequency of the first peak if it is determined in the second determination procedure that the higher harmonic wave is absent and outputs a respiration rate based on a fundamental frequency of the higher harmonic wave if it is determined in the second determination procedure that the higher harmonic wave is present.

11. A program for causing a computer to execute the biological detection method according to claim 10.
